(19) United States
(12) Patent Application Publication
(10) Pub. No.: US 2003/0190273 A1
(43) Pub. Date: Oct. 9, 2003

(54) Container liner for inhibiting decay of perishable items

(75) Inventors: Sumner A. Barenberg, Chicago, IL (US); Peter N. Gray, Chicago, IL (US); Gordon R. Putz, Chicago, IL (US); Michael D. Lelah, Chicago, IL (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Bernard Technologies, Inc.

(21) Appl. No.: 10/119,245

(22) Filed: Apr. 9, 2002

(51) Int. Cl. A61L 2/20

(52) U.S. Cl. 422/305; 422/5; 422/37

(57) ABSTRACT

A liner for placement in a container holding perishable food products to inhibit decay. The liner is a generally flat mat having a plurality of compartments containing two substances which generate antimicrobial gases. The liner has holes in the mat to facilitate drainage of liquid water past the liner for removal from the container.

### BACKGROUND OF THE INVENTION

This invention relates generally to preservation of perishable items, and in particular to a liner for placement in a container and which releases a gas to inhibit food spoilage and promote good product presentation of the items held in the container.

A substantial concern in the transport, storage, and display of perishable items, such as food, is maintaining freshness. Grocers bear a significant expense when fruits, vegetables, meat, and dairy products spoil before being sold. To preserve quality and extend shelf life, most supermarkets have one or more display case which holds perishable produce at reduced temperature. A mist of water is periodically dispensed to wash the produce and provide moisture which inhibits shrinkage. That water, along with water from melting ice placed alongside the produce, can become dirty or promote contamination and should be drained away for removal from the display case. Although the reduced temperature of the display case is somewhat effective in maintaining freshness, bacteria, mold, and fungus grow on the produce held in the case, producing odors and degrading shelf life.

An antimicrobial gas such as chlorine dioxide can be provided to control bacteria, mold, and fungus to reduce spoilage. Unfortunately, previous systems for delivering gas to perishable foods in a display case have been limited to dispensing only one type of gas, while it would be desirable to release two or more distinct gases, such as one gas to control microbial organisms and another gas to control mold. Previous systems have potential drawbacks of being hazardous for users to handle or being positioned to create a barrier which is impermeable to liquid such that the water fails to drain from the case.

### SUMMARY OF THE INVENTION

Among the several objects and features of the present invention may be noted the provision of a liner for placement in a container to inhibit spoilage of perishable items held in the container; the provision of such a liner which inhibits growth of mold; the provision of such a liner which inhibits growth of bacteria; the provision of such a liner which simultaneously releases two types of gas; the provision of such a liner which is safe to handle; the provision of such a liner which is lightweight; the provision of such a liner which does not retain water; and the provision of such a liner which permits liquid to drain away from the container.

In general, a device according to the present invention is capable of dispensing gas into an area which holds food products to inhibit spoilage of the food products. The device comprises a body sized and shaped for placement into the area and having compartments therein. A charge of a first substance is in one of the compartments, the substance being adapted to generate a first gas beneficial to food product storage. A charge of a second substance is in one of the compartments, the substance being adapted to generate a second gas beneficial to food product storage. The second gas has a different chemical composition than the first gas.

In another aspect, a device according to the present invention is for placement in a product holding area holding food products to dispense gas which is beneficial to storage of the food products and to facilitate drainage of liquid water past the device for removal from the product holding area. The device comprises a generally flat mat having an upper surface and a lower surface. A plurality of compartments are in spaced relationship on the mat, each compartment holding a solid substance which is capable of generating a gas for release into the product holding area which is beneficial to storage of food products. A plurality of passages are in the mat extending from the upper surface of the mat to the lower surface and suitable for drainage of liquid therethrough.

In yet a further aspect, a liner of the present invention is for placement in an area which holds food products to dispense at least two distinct gases which are beneficial to storage of the food products and to facilitate drainage of liquid water past the liner for removal from the food holding area. The liner comprises a generally flat mat having an upper layer and a lower layer, the upper layer being generally permeable to gas and the lower layer being generally impermeable to gas. A plurality of compartments are in spaced relationship on the mat and positioned between the upper layer and the lower layer, each compartment holding a solid substance which is capable of generating a gas for release into the food product area which is beneficial to food product storage. The plurality of compartments include a first compartment containing a first substance for generating a first gas and a second compartment containing a second substance for generating a second gas. The second gas has a different composition than the first gas. A plurality of passages are in the mat positioned generally between the compartments and extending from the upper layer of the mat to the lower layer and suitable for drainage of liquid therethrough.

In still a further aspect, a device of the present invention is capable of dispensing gas into an area to control odors therein. The device comprises a generally flat mat having a plurality of compartments in spaced relationship on the mat. The mat is sized for placement into the area and has an upper layer and a lower layer attached together along spaced apart seams and unconnected between the seams to define the compartments between the upper layer and the lower layer. A charge of a first substance is in one of the compartments, the substance being adapted to generate a first gas which generally controls odors. A charge of a second substance is in one of the compartments, the substance being adapted to generate a second gas which generally controls odors. The second gas having a different chemical composition than the first gas.

Other objects and features of the present invention will be in part apparent and in part pointed out hereinafter.

### BRIEF DESCRIPTION OF THE DRAWINGS

FIG. 1 is a view in plan of a display case liner of the present invention;

FIG. 2 is a perspective view, partially broken away, of the liner placed in a display case;

FIG. 3 is a fragmentary section of the display case on the line 3-3 of FIG. 2;

FIG. 4 is an enlarged, fragmentary section of the liner on line 4-4 of FIG. 1;

FIG. 5 is a view in plan of a liner of a second embodiment; and

FIG. 6 is a section similar to FIG. 4 of a liner of a third embodiment.

Corresponding reference characters indicate corresponding parts throughout the views of the drawings.

### DETAILED DESCRIPTION OF THE PREFERRED EMBODIMENT

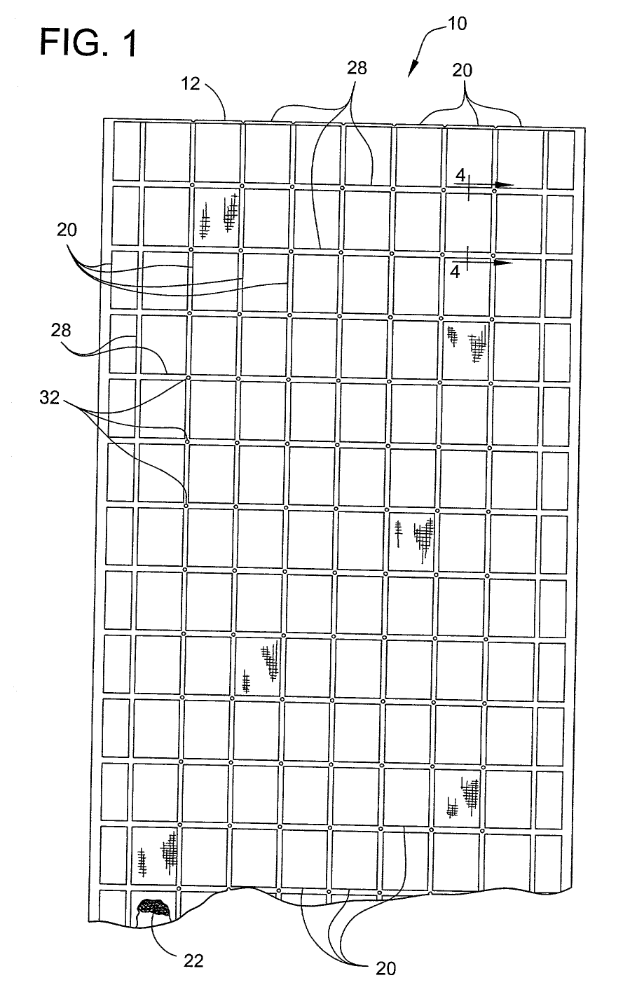

Referring now to the drawings and in particular to FIGS. 1-4, a liner according to the present invention is indicated generally at 10. The liner 10 is a device capable of dispensing gas and is sized and shaped to be placed in a product holding area containing perishable food products, such as fruit, vegetables, meat, or dairy products, to inhibit decay. Although the present invention has particular application to food product holding areas, it is envisioned that it could be used for other perishable products or for use in other areas to control odor.

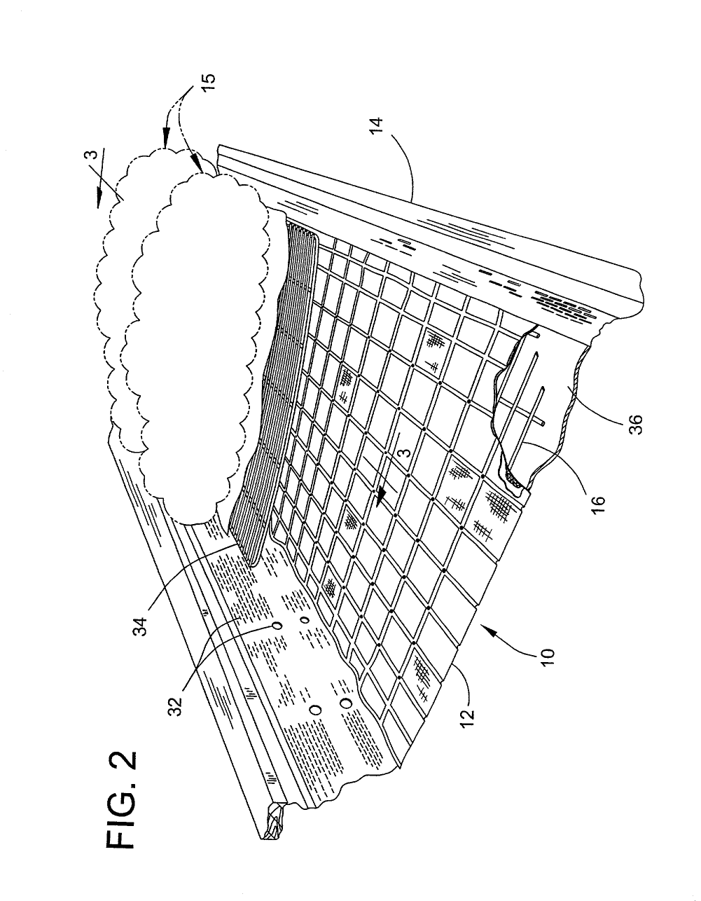

The liner 10 has a body comprising a generally thin, flat mat 12 of a shape configured to conveniently fit along an interior surface of a product holding area. The liner 10 has a generally rectangular shape corresponding to a rectangular shape of a typical produce display case 14 in a grocery store, and a size which preferably corresponds with a size of the display case. The liner 10 generally underlies the food products, which are indicated generally at 15 in FIG. 2, and overlies a drip pan 16 of the case. A typical size of the liner is 12.0 feet (366 cm) long by 2.5 feet (76 cm) wide, with a thickness generally less than one-eighth inch (0.3 cm). The liner 10 is formed of non-rigid materials such that the liner may be rolled up (not shown) for compact storage and portability when not being used. It is understood that the present invention could have a body with any shape or for other product holding areas and containers without departing from the scope of this invention. It may take a form (not shown) which is other than a mat and may occupy other positions in the product holding area. For instance, the invention might take the form of a bag placed next to the products in the product holding area.

The liner 10 has a plurality of compartments 20 for holding gas-generating chemicals 22. Each compartment 20 is generally rectangular in shape and is sealed to prevent escape of the chemicals 22 in the solid state while being partially permeable to liquid and gas. Each compartment 20 has dimensions preferably between 1 and 10 inches (2.5 to 25.4 cm) in either or both length and width directions. Exemplary sizes of each compartment 20 are 3 by 4 inches (7.6 by 7.6 cm), 2 by 2 inches (5.1 by 5.1 cm), and 4 by 4 inches (10.2 by 10.2 cm). As shown in FIG. 1, the liner 10 has ten columns of compartments 20. The outermost columns have compartments with a narrower width than the other columns. There may be any number of rows of compartments 20, depending on the length of the liner. The liner 10 may have perforations (not shown) positioned generally between the compartments 20 for adjusting the size of the liner. For a 12 foot long liner, there can be 360 total compartments (36 rows by 10 columns). It is understood that the liner 10 may have any number of compartments 20, including one, and each compartment may have any shape or size without departing from the scope of this invention.

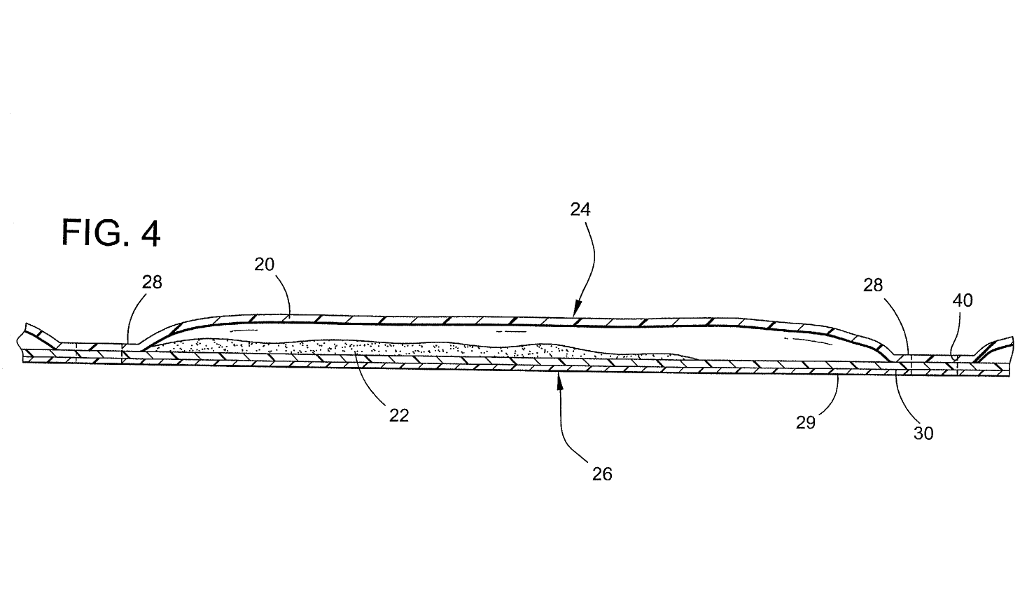

The liner 10 is formed of two or more continuous layers of materials, indicated generally at 24 and 26 (FIG. 4), which are sealed together at spaced apart seams 28. The layers 24, 26 are unconnected between the seams 28, with the compartments 20 being formed between the layers and bounded by the seams. The upper layer 24 of the liner is a suitable porous or microporous non-woven material which permits infusion of moisture, humidity, or light into the compartment 20 to activate the chemicals and allows release of generated gas from the compartment. An exemplary material is a hydrophobic polyethylene or polypropylene. The upper layer 24 may exhibit some limited absorbency but it does not contain any superabsorbent materials to soak up water which could result in the liner 10 becoming heavy and thereby degrading its ease of removability and lightweight, portable nature.

The lower layer 26 of the liner 10 includes a suitable barrier 29 which is impervious to water and has a low gas transmission rate. An exemplary material is polyester. As shown in FIG. 4, the lower layer 26 preferably has a laminate structure including a sheet 30 of Linear Low Density Polyethylene (LLDPE) on the inside of the barrier 29. The LLDPE of the lower layer 26 bonds to the non-woven material of the upper layer 24 upon application of heat. During manufacture, a square pattern of heated elements are pressed against the liner 10 to heat-seal the upper layer 24 to the lower layer 26 at the seams 28.

An advantage of the invention is unidirectional activation and release of gas. Moisture, humidity, or light can enter each compartment 20 only through the upper layer 24, and likewise gas generated within the compartment is released only through the upper layer. The liner 10 can therefore be positioned to face generally toward the perishable foods and/or sources of activating moisture to improve proximity thereto if desired. It is understood that the liner 10 can be configured to generate gas from both sides, i.e., both upper and lower layers 24, 26 can have equal gas, vapor, and liquid transmission characteristics without departing from the scope of this invention.

The gas-generating chemicals 22 can be any composition which generates and releases a gas upon exposure to an activating agent which may be water, humidity, and/or light. Preferably, the composition is an inorganic moisture activated composition (e.g., Microsphére® powder) as described in copending U.S. patent application Ser. No. 09/138,219 and U.S. Pat. No. 5,965,264, an organic moisture activated composition as described in U.S. Pat. Nos. 5,360,609, 5,631,300, 5,639,295, 5,650,446, 5,668,185, 5,695,814, 5,705,092, 5,707,739, 5,888,528, 5,914,120, 5,922,776, 5,980,826, and 6,046,243, or an inorganic light activated composition (e.g., Microlíte® powder) as described in U.S. Pat. No. 6,277,408 and copending U.S. patent application Ser. No. 09/448,927. For example, articles of the invention containing Microlíte® powder are activated by exposure to light, while articles containing Microsphére® powder are activated by exposure to humidity. All of the above-listed patents and applications are hereby incorporated by reference.

Each compartment 20 holds a charge of chemicals 22 for a controlled dose of active material gases to be dispensed in the display case 14. Typical quantity in each compartment 20 is between 0.4 and 0.8 grams. The quantity will depend on the size of the compartment 20, and some compartments may have no chemicals. The chemicals 22 can be in several solid forms, including, but not limited to, flowable powders, particles, granules, pellets, tablets, paste, spheres, gels, chips, and capsules.

In a preferred embodiment, the liner 10 has two or more solid substances for simultaneously releasing two or more types of gases. At least two distinct chemicals 22 are held in the compartments 20 and may be distributed among the compartments as desired. In the preferred embodiment, the outermost eight of ten columns (FIG. 1) of compartments 20 hold chemicals 22 which generate chlorine dioxide (ClO₂), known to those skilled in the art for its antimicrobial and odor neutralizing abilities. The innermost two columns of compartments 20 hold chemicals 22 for generating carbon dioxide (CO₂), known to those skilled in the art for its anti-mold, spoilage reduction, and odor control capabilities. Other arrangements are possible without departing from the scope of this invention. Although the chemicals 22 are segregated by type such that each compartment 20 produces either ClO₂ or CO₂, it is understood that chemicals to produce two or more gases could be mixed within the same compartments. Further, chemicals for producing a variety of gases other than ClO₂ or CO₂ which control microorganisims, reduce spoilage, or neutralize odors may be included without departing from the scope of this invention. One such gas that is effective in extending produce shelf life is sulfur dioxide (SO₂).

In addition to generating gas, the liner 10 may hold a charge of a third substance suitable for absorbing or adsorbing gas. Such absorption or adsorption increases shelf life by lessening ripening and decay. Exemplary materials are an ethylene scavenger, such as potassium permanganate, which absorbs ethylene from ripe fruit, or zeolite crystals, which absorb all gases. The liner 10 can therefore simultaneously generate, release, and absorb different gases. The gas absorption substance may replace one or more of the gas generating chemicals 22 in the liner 10 without departing from the scope of this invention.

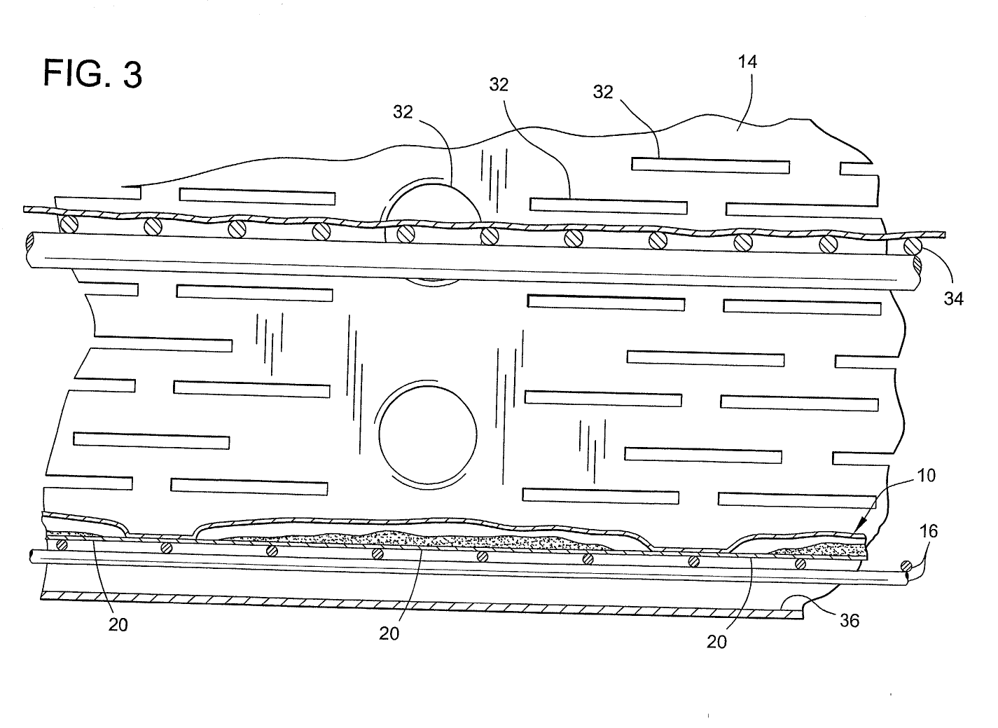

The liner 10 is shown installed in a typical display case 14 in FIGS. 2 and 3. The display case has a number of sidewall air holes 32 for providing air circulation. The food products 15 sit upon a display grate 34 which is spaced above the drip pan 16. The liner 10 is laid upon the drip pan 16 and is preferably sized to exactly fit the drip pan. Water which flows onto the liner 10 is delivered to a bottom tray 36 beneath the drip pan 16 and which is connected to a drain (not shown). It is understood that the liner 10 could be placed at any location in the display case 14, including the bottom tray 36, other surface (not shown) below the display grate 34, or may only partially cover the drip pan 16. The liner 10 could also be located along an upper surface or side of a container.

The liner 10 has a plurality of passages 40 extending from the upper layer 24 to the lower layer 26 to permit liquid to drain away from the display case 14. The passages 40 preferably are circular holes formed in the mat 12 generally at corners between adjacent compartments 20. The passages 40 are positioned in the seams 28 such that integrity of the compartments 20 is maintained. Although some of the water is absorbed by the upper layer 24 of non-woven material and/or by the chemicals 22, most flows through the passages 40.

In operation, the liner 10 is placed in a produce display case 14 where it receives exposure to an activating mechanism such as humidity, water, and/or light. That triggers the chemicals 22 held in the compartments 20 to begin generating gas. At least two distinct gases are provided, such as ClO₂ and CO₂, which act together to inhibit decay of the produce. The gases are distributed within the display case by air circulation. Typically, a mist of water is periodically dispensed on the produce to wash and provide moisture which inhibits shrinkage. That water, along with water from melting ice alongside the produce, flows onto the liner 10. Most of the water drains through the passages 40 and into the tray 36. Some of the water is absorbed by the material of the upper layer 24 and/or by the chemicals 22. The liner 10 is periodically replaced. An effective lifetime of the liner during normal use is about one month, although duration depends on the quantity of chemicals 22 initially charged in the compartments 20, the rate of exposure to the activating mechanism, and the temperature environment in which the liner is held (higher temperatures increase gas generation).

Significantly, the liner 10 provides a controlled, triggered release of gas when exposed to the activating mechanism, and does not continue to generate gas when no longer exposed. The liner is safe and non-toxic for handling or when in storage. Users may freely handle the liner without hazard.

The present invention can be used in other applications where it is desired to control odors, including household, industrial, and agricultural applications. For example, the liner 10 may be placed inside a container for shipment or transport of perishable items. Further, the liner may be placed in a kitchen, bathroom, underneath a sink, or in various places relating to the funeral home industry including inside of human caskets.

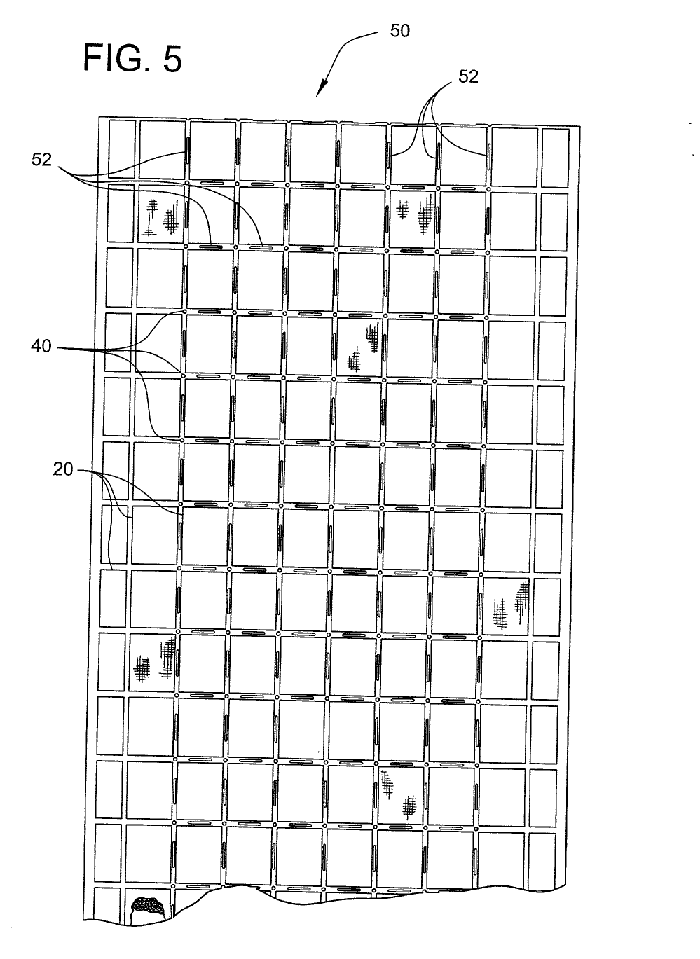

A second embodiment 50 of the invention (FIG. 5) is configured for improved drainage of water. The liner 50 includes elongate slits 52 between the compartments 20 and between the passages 40. Like the passages 40, the slits 52 are in the heat sealed seams 28 so as to maintain integrity of the compartments 20 and prevent leaks of powder chemicals 22. The slits 52 have a suitable length which may range from 10 to 80% of the length of the adjacent compartment 20. The slits 52 may be in both length and width directions, as shown on FIG. 5, or alternatively may be in only one direction or at a random pattern.

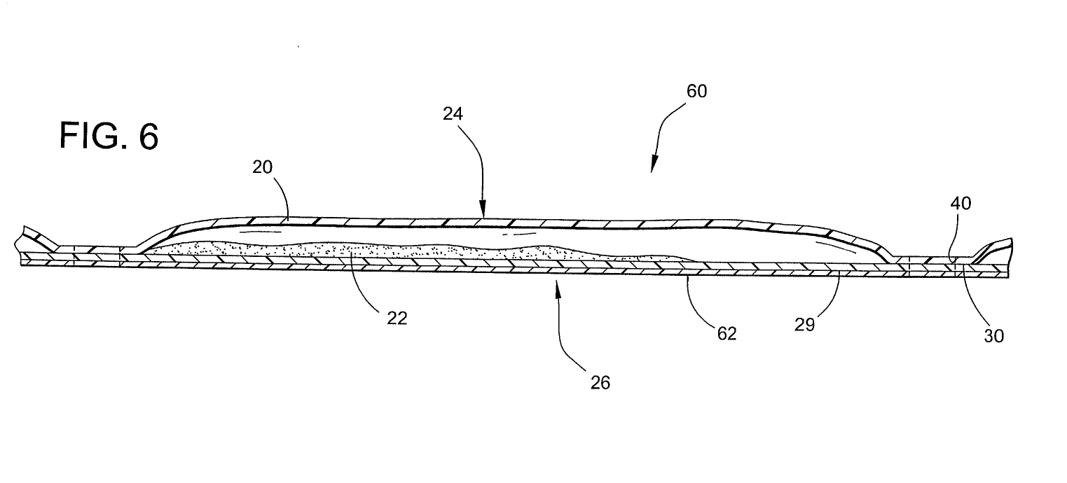

A third embodiment 60 of the liner is shown in FIG. 6. The lower layer 26 of the liner 60 is a laminate having a third layer 62, positioned beneath the barrier 29. The third layer 62 comprises polyethylene which protects the polyester of the barrier 29 from scratching. In particular, the third layer 62 protects any printing or identifying markings on the polyester layer when the liner 10 is placed on the drip pan 16 or any abrasive surface. It is understood that a liner 10 having a lower layer 26 with any number of layers of materials, including one, does not depart from the scope of this invention.

In view of the above, it will be seen that the several objects of the invention are achieved and other advantageous results obtained.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above without departing from the scope of the invention, it is intended that all matter contained in the above description and shown in the accompanying drawings shall be interpreted as illustrative and not in a limiting sense.

What is claimed is:

1. A device capable of dispensing gas into an area which holds food products to inhibit spoilage of the food products, the device comprising:
a body sized and shaped for placement into said area and having compartments therein;
a charge of a first substance in one of said compartments, the substance being adapted to generate a first gas beneficial to food product storage; and
a charge of a second substance in one of said compartments, the substance being adapted to generate a second gas beneficial to food product storage, the second gas having a different chemical composition than the first gas.

2. A device as set forth in claim 1 wherein said body comprises a generally flat mat for placement in said food product area in a position generally underlying the food products.

3. A device as set forth in claim 2 wherein the mat comprises an upper layer and a lower layer attached together along spaced apart seams and unconnected between the seams to define the compartments.

4. A device as set forth in claim 3 wherein each of said compartments holds either said first substance or said second substance.

5. A device as set forth in claim 3 further comprising a charge of a third substance, the third substance being adapted to absorb gas.

6. A device as set forth in claim 3 wherein the upper layer is generally permeable to gas and the lower layer is generally impermeable to gas.

7. A device as set forth in claim 3 wherein the mat has at least one passage extending from the upper layer to the lower layer for passage of liquid through the mat.

8. A device as set forth in claim 7 wherein said passage is located at a seam.

9. A device as set forth in claim 7 wherein said passage is a circular hole.

10. A device as set forth in claim 7 wherein said passage is an elongate slot.

11. A device as set forth in claim 2 wherein the first gas is chlorine dioxide and the second gas is carbon dioxide.

12. A device as set forth in claim 2 wherein each of said first and second substances is activated to generate gas by exposure to one of water, humidity, or light.

13. A device as set forth in claim 1 wherein said body has at least one passage extending through the body for passage of liquid.

14. A device for placement in a product holding area holding food products to dispense gas which is beneficial to storage of the food products and to facilitate drainage of liquid water past the device for removal from the product holding area, the device comprising:
a generally flat mat having an upper surface and a lower surface;
a plurality of compartments in spaced relationship on the mat, each compartment holding a solid substance which is capable of generating a gas for release into the product holding area which is beneficial to storage of food products; and
a plurality of passages in the mat extending from the upper surface of the mat to the lower surface and suitable for drainage of liquid therethrough.

15. A device as set forth in claim 14 wherein said passages are positioned in the mat generally between the compartments.

16. A device as set forth in claim 15 wherein said passages are circular holes.

17. A liner for placement in an area which holds food products to dispense at least two distinct gases which are beneficial to storage of the food products and to facilitate drainage of liquid water past the liner for removal from the food holding area, the liner comprising:
a generally flat mat having an upper layer and a lower layer, the upper layer being generally permeable to gas and the lower layer being generally impermeable to gas;
a plurality of compartments in spaced relationship on the mat and positioned between the upper layer and the lower layer, each compartment holding a solid substance which is capable of generating a gas for release into the food product area which is beneficial to food product storage, said plurality of compartments including a first compartment containing a first substance for generating a first gas and a second compartment containing a second substance for generating a second gas, the second gas having a different composition than the first gas; and
a plurality of passages in the mat positioned generally between the compartments and extending from the upper layer of the mat to the lower layer and suitable for drainage of liquid therethrough.

18. A device capable of dispensing gas into an area to control odors therein, the device comprising:
a generally flat mat having a plurality of compartments in spaced relationship on the mat, the mat being sized for placement into said area and having an upper layer and a lower layer attached together along spaced apart seams and unconnected between the seams to define the compartments between the upper layer and the lower layer;
a charge of a first substance in one of said compartments, the substance being adapted to generate a first gas which generally controls odors; and
a charge of a second substance in one of said compartments, the substance being adapted to generate a second gas which generally controls odors, the second gas having a different chemical composition than the first gas.
